(19) United States
(12) Patent Application Publication
(10) Pub. No.: US 2024/0285867 A1
(43) Pub. Date: Aug. 29, 2024

(54) MEDICATION DELIVERY DEVICE WITH DOSE BUTTON

(71) Applicant: Eli Lilly and Company, Indianapolis, IN (US)

(72) Inventor: Jaime Ray ARNETT, Fishers, IN (US)

(21) Appl. No.: 18/571,865

(22) PCT Filed: Jun. 29, 2022
(86) PCT No.: PCT/US22/35402

§ 371 Date: Dec. 19, 2023

Related U.S. Application Data
(60) Provisional application No. 63/217,465, filed on Jul. 1, 2021.

(51) Int. Cl. A61M 5/315

(52) U.S. Cl. CPC A61M 5/31553; A61M 5/31568; A61M 5/31585

(57) ABSTRACT

Medication delivery devices are provided having a dose delivery button. A dose button includes a contact interface to enhance friction between a user's finger when operating the device to promote substantially axial translation of a dose button to deliver a dose of medication. The contact interface may be formed of a different material with a lower coefficient of kinetic friction relative to the skin and/or a lower Young's modulus than that of the dose button. The contact interface may have a smaller lateral dimension than the dose button. The delivery device may have an actuator cover coupled to the contact interface.

### FIELD

Disclosed embodiments are related to medication delivery devices and related methods of use.

### BACKGROUND

Patients suffering from various diseases must frequently inject themselves with medication. To allow a person to conveniently and accurately self-administer medicine, a variety of devices broadly known as pen injectors or injection pens have been developed. Generally, these pens are equipped with a cartridge including a piston and containing a multi-dose quantity of liquid medication. A drive member is movable distally to advance the piston in the cartridge to dispense the contained medication from an outlet at the distal cartridge end, typically through a needle.

Many pen injectors and other medication delivery devices utilize mechanical systems in which members rotate and/or translate relative to one another in a manner proportional to the dose delivered by operation of the device. The administration of a proper amount of medication requires that the dose delivered by the medication delivery device be accurate.

### SUMMARY

In some embodiments, a medication delivery device includes a housing disposed about a longitudinal axis and having an outlet. A rotating dose member is rotatable about the longitudinal axis relative to the housing during dose setting. A dose button is configured to be translatable along the longitudinal axis in an axial direction relative to the housing to activate a dose dispensing mode in which medication is dispensed out of the outlet. The dose button includes a proximal surface. A contact interface is disposed proximal to and configured to contact the proximal surface of the dose button. The contact surface has a proximal surface. The contact interface and the dose button have a coaxial relationship. The proximal surface of the dose button includes a first dimensional parameter. The proximal surface of the contact interface includes a second dimensional parameter that is smaller than the first dimensional parameter. The second dimensional parameter is sized to enhance on-center axial loading of the contact surface during dose delivery and inhibit any axial loading on the rotating dose member.

In some embodiments, a medication delivery device comprises a housing having an outlet, a dose button, and a data module. The dose button is configured to be translatable in an axial direction relative to the housing to activate a dose dispensing mode in which medication is dispensed out of the outlet. The dose button includes a proximal surface. The data module is configured to measure a property in the dose dispensing mode. The data module includes a contact interface, and the data module is operatively coupled to the dose button. A first lateral dimension is measured across the proximal surface of the data module in a lateral direction is greater than a second lateral dimension measured across a proximal surface of the contact interface in the lateral direction. The lateral direction is perpendicular to the axial direction.

In some embodiments, a method of delivery medication comprises applying an axial force to a contact interface operably coupled to a proximal surface of a dose button, displacing the dose button relative to a housing in an axial direction, and activating a dose dispensing mode in which a medication is dispensed out of an outlet with the displacement of the dose button. A first lateral dimension measured across the proximal surface of the dose button in a lateral direction is greater than a second lateral dimension measured across a proximal surface of the contact interface in the lateral direction, the lateral direction being perpendicular to the axial direction.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a front view of one embodiment of a medication delivery device having a contact interface in accordance with some aspects;

FIG. 2 is an enlarged portion of the medication delivery device of FIG. 1;

FIG. 3 is a front view of another embodiment of a medication delivery device having a data module and a contact interface in accordance with some aspects;

FIG. 4 is a partial cross-section of the medication delivery device from FIG. 3 taken along line 4-4, in which the data module and contact interface are shown in cross-section;

FIG. 5 is partial cross-section of another embodiment of a medication delivery device, in which the data module and contact interface are shown in cross-section;

FIG. 6 is partial front view of yet another embodiment of a medication delivery device having a data module, a contact interface, and an actuator cover in accordance with some aspects;

FIG. 7 is a top view of an actuator cover according to some embodiments;

FIG. 8 is a top view of a contact interface according to some embodiments;

FIG. 9 is a perspective view a contact interface according to some embodiments;

FIG. 10 is a cross-sectional view of a contact interface according to some embodiments;

FIG. 11 is a top view of a cover according to some embodiments, with an arcuate sidewall;

FIG. 12 is a top view of a cover according to some embodiments, with a U-shaped sidewall;

FIG. 13 is a side view of a contact interface with a tapered sidewall according to some embodiments coupled to a data module;

FIG. 14 is a top view of a cover according to some embodiments, with a plurality of parallel ribs; and

FIG. 15 is a perspective view of one embodiment of a medication delivery device having a contact interface in accordance with some aspects.

### DETAILED DESCRIPTION

It should be understood that aspects are described herein with reference to certain illustrative embodiments and the figures. The illustrative embodiments described herein are not necessarily intended to show all aspects, but rather are used to describe a few illustrative embodiments. Thus, aspects are not intended to be construed narrowly in view of the illustrative embodiments. In addition, it should be understood that certain features disclosed herein might be used alone or in any suitable combination with other features.

Medical delivery devices can be arranged such that operation of the device involves a user actuating a dose button, which may cause medication to be delivered out of an injection needle at an outlet end of the device. In some embodiments, a user actuates a dose button by applying an axial force to the dose button, e.g., by pushing on the dose button. In some embodiments, a user actuates a dose button by actuating a device actuator that is separate and distinct from the dose button. Actuation of the device actuator may then cause axial force to be applied to the dose button, e.g., via intermediate components between the device actuator and the dose button. In some embodiments, the device actuator is actuated by a user pushing on the device actuator. The device may include any number of components that are operably linked between the dose button and the injection needle to enable the outflow of medication in response to actuation of the dose button.

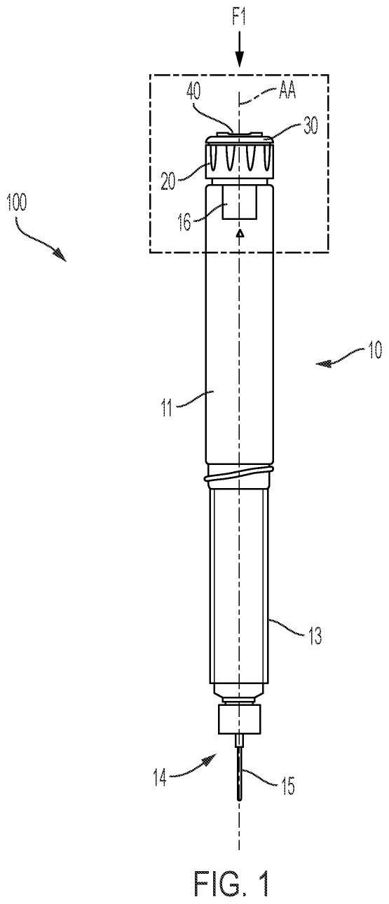

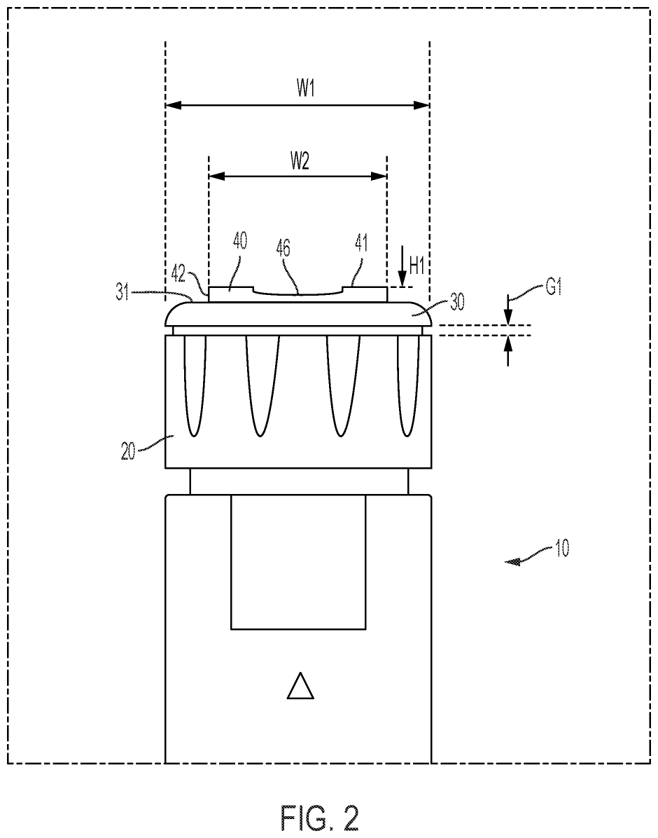

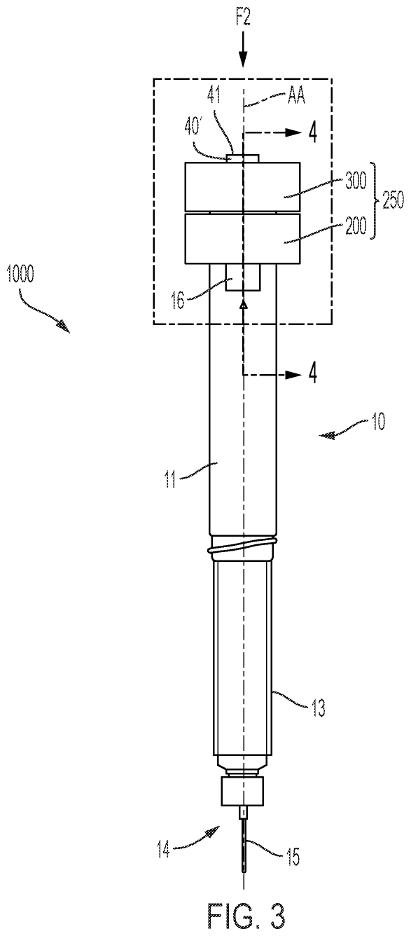

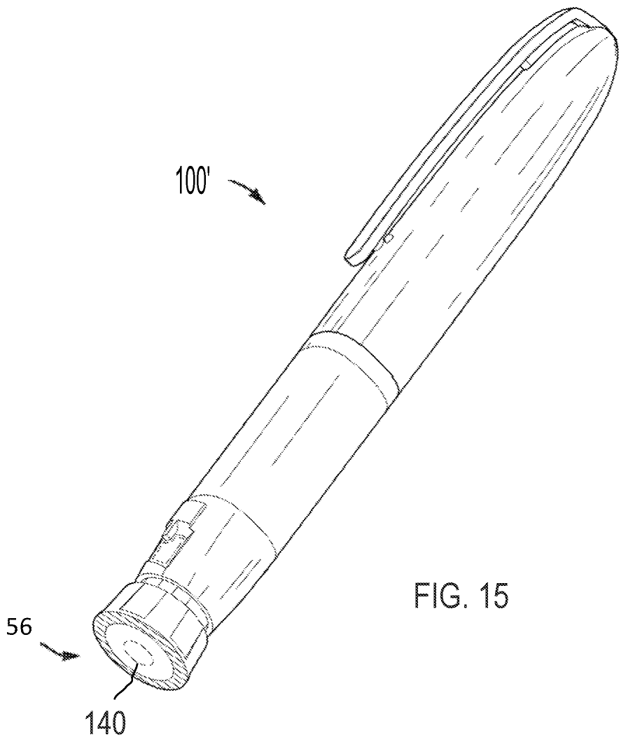

The inventors have recognized that such medical delivery devices may benefit from the inclusion of one or more features, such as ergonomic features, that may help to facilitate use. For example, such features may help to promote application of axial force to the dose button at or closely along the longitudinal axis of the device and/or may help to reduce unintended application of force relatively farther way from the longitudinal axis to a rotating component during dose delivery, such as, for example, a rotating dose member, the dose button, or a dose data module coupled to the dose button. This may be beneficial for dose buttons having proximal surfaces with larger surface areas and/or data modules that attach to the dose button that have proximal surfaces with larger surface areas. “Rotating dose member” may refer to a component that is rotated about the longitudinal axis relative to the device housing by a user during dose setting and/or may rotate automatically in the direction opposite the dose setting direction about the longitudinal axis relative to the device housing during dose dispensing. Depending on the device, the rotating dose member may be a rotatable collar as shown, for example, in FIGS. 1-2, a data module that is attached to the collar as shown as shown, for example, in FIGS. 3-5, or an integrated dose button/collar single component like in the KwikPen™ provided by Eli Lilly and Company (Indianapolis, Indiana) (an embodiment shown in FIG. 15). When such rotating dose member component rotates during dose dispensing, features disclosed herein help avoid applying a force to the rotating component that may cause drag during rotation may not be preferred by the patient or may impact sensing accuracy in the case of using a data module. In some cases, such rotating dose member component is rotationally fixed during dose dispensing, the features disclosed herein that avoid applying a force to such dose rotating component that may cause rotation that may not be preferred by the patient or may impact sensing accuracy in the case of using a data module.

According to one aspect, a medication delivery device may be provided with a contact interface that is configured to be contacted by a user in order to actuate the medication delivery device. In some embodiments, the contact interface may have a smaller contactable surface area than the dose button, such that a lateral dimension of the dose button taken along a lateral direction may be greater than a lateral dimension of the contact interface taken along the lateral direction. In this way, the contact interface may serve as a guide for a user's finger (or any other suitable appendage or tool used to actuate the device) to apply an axial force to the dose button approximately along the longitudinal axis of the device in order to dispense a dose.

According to another aspect, the contact interface may be formed of a material to improve contact between a user's finger and the dose button. In some embodiments, improved contact between the user's finger and the dose button may help to reduce unintended application of non-axial force to the dose button.

According to some embodiments of the present technology, the contact interface may be both smaller than the dose button in a lateral dimension (as will be described in further detail below) and may be formed of a material capable of greater friction with the user's finger when compared to the dose button. Of course, embodiments where the contact interface is laterally smaller than the dose button, but formed of the same material, and/or embodiments where the contact interface is similarly sized to the dose button, but is formed of a friction-enhancing material, are also contemplated.

In some embodiments, the contact interface may be formed of a material which may be more compliant (e.g., softer) than the dose button. In some embodiments, as a result, the dose dispensing operation may be more ergonomic and/or comfortable for the user when applying an axial force to the contact interface. In some embodiments, the contact interface may be formed of any suitable biocompatible polymer, plastic, rubber, thermoplastic material, composite, or any other moldable material. In some embodiments, the contact interface may be formed of an elastomeric material. In some embodiments, the contact interface may be formed of any suitable material, including, but not limited to, polyisoprene, natural rubbers, polybutadiene, neoprene, polyisobutylene, polyurethanes, chloroprene, butyl rubbers, nitrile rubbers, polyacrylic rubbers, fluoroelastomers, ethylene vinyl acetate, synthetic rubbers such as ethylene-propylene-diene monomer rubber, block co-polymers, polysiloxanes, thermoplastic urethanes, thermoplastic rubbers, polyurethanes (including thermoplastic polyurethanes), polypropylene, polyethylene, ethylene vinyl alcohol, polyamide, polychlorotrifluoroethylene, cyclic olefin copolymer, polycarbonate, ethylene vinyl acetate, polyvinyl chloride, polyvinylidene chloride, polystyrene, polyethylene terephthalate, poly di-methyl siloxane, thermoplastic elastomer, polymethyl methacrylate, liquid silicone members, textiles, composites, or any other suitable material or combinations thereof. It should be appreciated the current disclosure is not limited by the material composition of the contact interface.

Accordingly, a Young's modulus (or any other suitable measure of elasticity, including but not limited to, storage modulus, bulk modulus, tensile modulus) of the contact interface may be lower than a Young's modulus of the dose button. In some embodiments, the Young's modulus of the contact interface may be at least 1 kPa, 5 kPa, 10 kPa, 20 kPa, 25 kPa, 50 kPa, 75 kPa, 100 kPa, 200 kPa, 300 kPa, 500 kPa, 750 kPa, 1 MPa, 1.2 MPa, 1.5 MPa, 2 MPa, 3 MPa, or any other suitable modulus. In some embodiments, the Young's modulus of the contact interface may be less than or equal to 3 MPa, 2 MPa, 1.5 MPa, 1.2 MPa, 1 MPa, 750 kPa, 500 kPa, 300 kPa, 200 kPa, 100 kPa, 75 kPa, 50 kPa, 25 kPa, 20 kPa, 20 kPa, 10 kPa, 5 kPa, 1 kPa, or any other suitable modulus. Combination of the foregoing ranges are also contemplated. For example, in some embodiments, the Young's modulus of the contact interface may be between 1 kPa and 3 MPa, 10 kPa and 100 kPa, 5 kPa and 1 MPa, 25 kPa and 1 MPa, 50 kPa and 2 MPa, 100 kPa and 1 MPa, or any other suitable range of moduli. It should be appreciated that any suitable material with any Young's modulus may be employed, as the current disclosure is not so limited.

It should also be appreciated that in some embodiments, the contact interface may be formed of more than one material. In some embodiments, a first material may be more compliant and may enhance frictional contact between the user's finger and the interface, as described earlier, and a second material may provide rigidity. In some embodiments, the second material may allow a substantial proportion of a force applied to the contact interface to be transferred to the dose button without significant absorption of the force by the contact interface.

In some embodiments, the dose button may be formed of a rigid material such that a user-applied force to a push surface of the dose button may be substantially translated to the mechanical components of the device to deliver a dose of medication. Accordingly, the dose button may be formed of any suitable material, including, but not limited to polypropylene, cyclic olefin copolymer, polymethyl methacrylate, copolyester, polyethylene terephthalate, polycarbonate, polystyrene, high density polyethylene, metals, composites, or any other suitable material of combinations thereof. It should be appreciated the current disclosure is not limited by the material composition of the dose button.

In some embodiments, a Young's modulus of the dose button may be greater than a Young's modulus of the contact interface. In some embodiments, the Young's modulus of the dose button may be at least 1.2, 1.4, 1.5, 2, 2.5, 3, 4, 5, 7, 10, 15, 20, 25, 30, 35, 40, 45, 50, 100, 120, 150, 200, 250, 300, 350, 400, 500, 1000, 1100, 1200, 1500, 2000, 2200, 2500, 3000 or 5000 times greater than the Young's modulus of the contact interface. In some embodiments, the Young's modulus of the dose button may be less than or equal to 5000, 3000, 2500, 2200, 2000, 1500, 1200, 1100, 1000, 500, 400, 350, 300, 250, 200, 150, 120, 100, 50, 45, 40, 35, 40, 25, 20, 15, 10, 7, 5, 4, 3, 2.5, 2, 1.5, 1.4, or 1.2 times greater than the Young's modulus of the contact interface. Combinations of the foregoing ranges are also contemplated. For example, in some embodiments, the Young's modulus of the dose button may be between 1.2 and 5000, 10 and 5000, 100 and 2000, 100 and 1000, 1000 and 5000, or 25 and 2500, times greater than the Young's modulus of the contact interface. In some embodiments, the Young's modulus of the dose button may be substantially equal to the Young's modulus of the contact interface. It should be appreciated that the Young's modulus of the dose button may be any suitable proportion of the Young's modulus of the contact interface, as the current disclosure is not so limited.

According to some embodiments, the contact interface may be formed of a material which may enhance friction during contact between the user's finger and the contact interface. Accordingly, a coefficient of kinetic friction between the contact interface and the user's finger may be greater than a coefficient of kinetic friction between the dose button and the user's finger. In some embodiments, the coefficient of kinetic friction between the contact interface and a user's finger may be at least 5%, 10%, 12%, 15%, 20%, 25%, 30%, 33.33%, 35%, 40%, 45%, 50%, 60%, 66.67%, 75%, 80%, 90%, 100%, 120%, 140%, 150%, 160%, 175%, 200%, 225%, 250%, 275%, 300%, 400%, or any other suitable percentage greater than the coefficient of kinetic friction between the dose button and a user's finger. In some embodiments, the coefficient of kinetic friction between the contact interface and a user's finger may be less than or equal to 400%, 300%, 275%, 250%, 225%, 200%, 175%, 160%, 150%, 140%, 120%, 100%, 90%, 80%, 75%, 66.67%, 60%, 50%, 40%, 35%, 33.33%, 30%, 25%, 20%, 15%, 12%, 10%, 5%, or any other suitable percentage greater than the coefficient of kinetic friction between the dose button and a user's finger. Combination of the foregoing ranges are also contemplated. For example, in some embodiments, the coefficient of kinetic friction between the contact interface and a user's finger may be 5% to 400%, 10% to 200%, 50% to 400%, 33.33% to 300%, 33.33% to 66.67%, or 100% to 200% greater than the coefficient of kinetic friction between the dose button and a user's finger. In some embodiments, the coefficient of kinetic friction between the user's finger and the dose button may be substantially similar to the coefficient of kinetic friction between the user's finger and the contact interface. It should be appreciated that any suitable proportion between the coefficient of kinetic friction between the user's finger and the dose button and the coefficient of kinetic friction between the user's finger and the contact interface may be employed, as the current disclosure is not so limited.

It should be appreciated that the contact interface may be sized and/or shaped in any suitable manner to promote axial translation of the dose button when the device is in a dose dispensing mode. Accordingly, the contact interface may have ergonomic features, as described in further detail below, including, but not limited to tapered edges, protrusions, one or more recesses, or any other suitable feature or combination of features. In this way, the contact interface may serve to center and align the user's finger with an axial direction of the dose button.

In some embodiments, the contact interface may extend a height of the device measured along an axial direction. It should be appreciated that the contact interface may have any suitable height measured along the axial direction of the device. In some embodiments, the contact interface may protrude from a surface of a dose button or from a portion of a data module installed on a medication delivery device.

According to some embodiments of the present disclosure, the contact interface may include a lateral dimension which may be less than a lateral dimension of a dose button, where the lateral dimension is measured along a plane normal to the axial direction of the device.

It should be appreciated that the contact interface may include any suitable shape or structure, including, for example, tapered, rounded, chamfered, and/or curved sidewalls, as will be described in further detail below. In some embodiments, the shape of the sidewall of the contact interface may help to translate off-axial forces applied by the user to on-axial forces, to assist with actuation of the dose button.

In some embodiments, the contact interface may include one or more surface features, such as protrusions (e.g., ribs) or recesses. In some embodiments, such surface features may serve to reduce lateral movement of the user's finger on the friction surface. The contact interface may have any number of features or combinations of features, as the present disclosure is not so limited.

Devices described herein may further comprise a medication, such as for example, within a reservoir or cartridge contained within a housing of the device, as described in further detail below. The term “medication” refers to one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, oxyntomodulin analogs, oxyntomodulin derivatives, therapeutic antibodies and any therapeutic agent that is capable of delivery by the above device. The medication as used in the device may be formulated with one or more excipients. The device is operated in a manner generally as described above by a patient, caregiver or healthcare professional to deliver medication to a person.

In some embodiments, a dose button may be attached to a component of the medication delivery device by being directly positioned on, received within, integral with, or otherwise connected to, the component. Connections may include, for example, connections formed by frictional engagement, splines, a snap or press fit, sonic welding or adhesive.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein. For example, while the medication delivery device is described in the form of a pen injector, the medication delivery device may be any device which is used to set and to deliver a dose of a medication, such as pen injectors, autoinjectors, bolus injectors, infusion and syringes. The medication may be any one of a type that may be delivered by such a medication delivery device. The medication delivery device may be a reusable device capable of receiving a replaceable and disposable cartridge of medication or may be an entirely disposable device with a prefilled reservoir of medication.

FIGS. 1-2 show a medication delivery device 100 according to some embodiments. The medication delivery device 100 (hereinafter “device”) includes an elongated pen-shaped housing 10, including a distal portion 13 and a proximal portion 11, wherein the terms “distal” and “proximal” are used relative to the application of force from the patient, making the needle end the distal end of the device and the actuator end of the device the proximal end. In some embodiments, the distal portion 13 may include a reservoir or cartridge (not shown) configured to hold a medicinal fluid to be dispensed through an outlet 14 during a dispensing operation. The outlet 14 of distal portion 13 may be equipped with an injection needle 15. In some embodiments, the injection needle 15 may be removable from the housing 10. In some embodiments, the injection needle 15 may be replaced with a new injection needle after each use. In other embodiments, the housing 10 may be reusable, and the cartridge may be configured to be replaced. The device 100 may also include a pen cap (not shown) to cover or otherwise protect the injection needle 15 of the device 100.

In some embodiments, the proximal portion 11 of the housing 10 may include a drive member (not shown), which may be a screw or any other suitable driving mechanism, configured to transfer force from a user (e.g., a patient) to a piston located in the distal portion 13 to force a preset dose of medicinal fluid out of the needle 15. Accordingly, the drive member may be axially moveable relative to the housing 10.

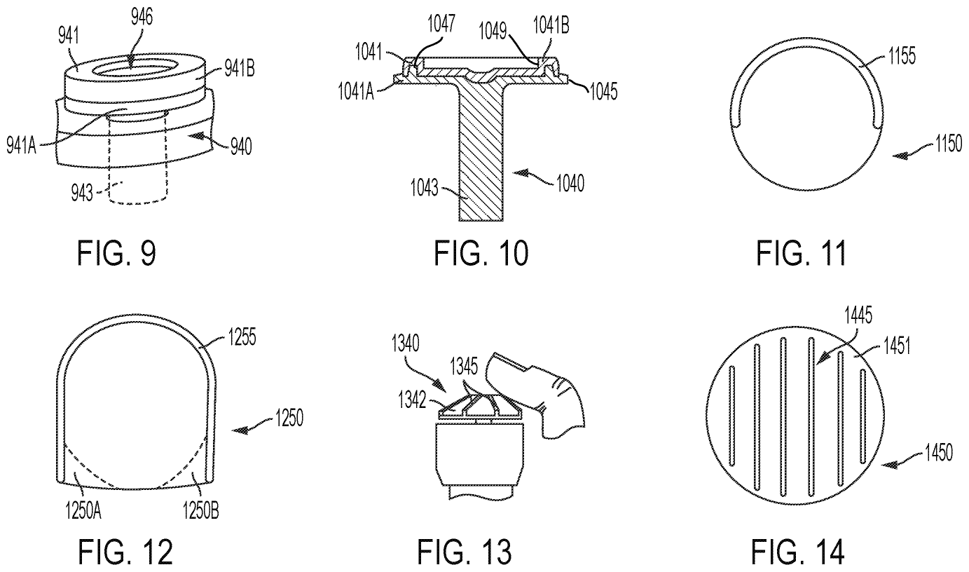

In some embodiments, a device 100 may include a rotatable dose select collar 20 (hereinafter referred to as “rotatable collar”), a dose button 30, and a contact interface 40 located at one end of the proximal portion 11 of the housing 10. While the rotatable collar 20, the dose button 30, and the contact interface 40 are shown in FIG. 1 to be coaxially located with respect to a longitudinal axis AA, other arrangements of the rotatable collar 20, dose button 30, and contact interface 40 are also contemplated, as the present disclosure is not so limited. The contact interface 40 may be coupled and/or fixed to the dose button 30 such that pressing (e.g., axial translation) the contact interface 40 towards the distal portion 13 may also axially translate the dose button 30 along an axial direction (e.g., along longitudinal axis AA). In some embodiments, the dose button 30 may be mechanically coupled to the drive member of the proximal portion 11 such that depression of the contact interface 40 may result in ejection of fluid from the distal portion 13, as previously described. In some embodiments, the dose button is rotatable relative to the housing, or in other words, free spinning. The contact interface 40 may include a push surface 41 to allow a user to apply a distally directed force F1 to the contact interface 40 (and subsequently, the dose button 30) to operate the device 100. In some embodiments, the push surface 41 may include a contact interface recess 46, as shown in FIG. 2. In some embodiments, a contact interface recess may help to align a user's finger centrally on the contact interface 40. The force F1 may be an on-centered force. The contact interface 40 is configured to inhibit an off-center axial loading from applied force F1 on the rotating dose member. The shape of the contact interface recess 46 may form a circular shape that is coaxial with the axis AA, such as, for example, the recess 946 shown in FIG. 9, while other shapes are contemplated, such as, for example, hexagonal, rectangular, or shapes suitable to increase purchase of the patient's finger during operation. A recess like the contact interface recess 46 may also be incorporated directly into any of the embodiments of the contact interface, such as, e.g., the contact interface 40′.

In some embodiments, the device 100 may be operable in a dose setting mode. In some embodiments, the rotatable collar 20 may be rotated in one of a clockwise or counterclockwise direction to adjust and select the dosage (e.g., volume of medication to be injected). In some embodiments, the device 100 may be operable in a dose dispensing mode, in which the dose button 30 is axially translated relative to the rotatable collar 20 to deliver the preset dosage of medication to the patient through an injection needle 15. As discussed herein, the dose button 30 may be axially translated in response to a user pressing on a contact interface 40. During dose dispensing, the dose button 30 is depressed, while the rotatable collar rotates in the other of the clockwise or counterclockwise direction, that is the opposite direction from dose setting. In the device 100, the rotating dose member comprises the rotatable collar 20 that is rotatable about the longitudinal axis relative to the housing 10 during dose setting and may be rotatable about the longitudinal axis relative to the housing 10 during dose dispensing.

In some embodiments, in the dose setting mode of operation, the rotatable collar 20 may be rotated relative to housing 10 to set a desired dose to be delivered by device 100. In some embodiments, the rotatable collar 20, the dose button 30, and the contact interface 40 may be rotatably fixed to one another during the dose setting mode of operation. In other words, rotation of the rotatable collar 20 may also cause the dose button 30 and the contact interface 40 to rotate. It should be appreciated that the present disclosure is not limited by the means or mechanisms to rotatably fix the rotatable collar 20, the dose button 30, and the contact interface 40 to one another during the dose setting mode. The contact interface may be rotationally fixed to the second portion of the data module.

After a user has completed setting a dose, the user may then actuate the device to cause axial translation of the dose button. Axial translation of the dose button may then trigger a dose dispensing mode.

In some embodiments, the dose button 30 may be axially translatable relative to the rotatable collar 20, which may be separated from the dose button 30 by a gap G1, as shown in FIG. 2. Axially translating the dose button 30 toward the rotatable collar 20 to reduce gap G1 may trigger the dose dispensing mode. In some embodiments, the rotatable collar 20 may rotate as the dose button 30 is axially translated toward the rotatable collar. In some embodiments, the rotatable collar and the dose button become rotationally uncoupled in the dose dispensing mode, such that the rotatable collar rotates relative to the dose button during dispensing of fluid.

It should be appreciated that the current disclosure is not limited by the coupling mechanism between the dose button 30 and the rotatable collar 20.

In some embodiments, rotating the rotatable collar 20 in a first direction may serve to increase the set dose, and rotating the rotatable collar 20 in a second opposite direction may serve to decrease the set dose. The rotatable collar 20 may be rotationally adjustable in pre-defined rotational increments corresponding to a minimum incremental increase or decrease of the set dose during the dose setting operation. The rotatable collar 20 may include a detent mechanism such that each rotational increment produces an audible and/or tactile “click.” For example, one increment or “click” may equal one-half or one unit of medication. In some embodiments, the set dose amount may be visible to the user via a series of dial indicator markings shown through a dosage window 16, as shown in FIG. 1.

Once a desired dose of medication fluid is set by rotating the rotatable collar 20, device 100 may be manipulated so the injection needle 15 properly penetrates, for example, a user's skin. The dose dispensing mode of operation may be initiated in response to an axial distal force (e.g., F1, as shown in FIG. 1) applied to the push surface 41 of the contact interface 40. The axial force F1 may be applied by the user directly to the contact interface 40 to axially translate the dose button 30, which may interact with a drive member of the medication delivery device to deliver the medication fluid to the user. In some embodiments, the dose dispensing mode of operation may be completed when the dose button 30 has returned to its zero-dose position. In some embodiments, the rotatable collar 20 may rotate relative to the housing 10 while the dose button 30 is rotationally stationary relative to the housing 10 during the dose dispensing mode.

It should be appreciated that while two distinct bodies are shown for the rotatable collar 20 and dose button 30, such as, for example, can be found in Ergo II pen provided by Eli Lilly and Company (Indianapolis, Indiana), in some embodiments of the device 100, the rotatable collar 20 and the dose button 30 may be integrally formed, such that a single body, which can be called a dose button, may be rotated relative to the housing 10 and rotationally fixed with the dose setting member to set a dose, and may be axially translated relative to the housing 10 (but is configured to rotate relative to the dose setting member) to dispense a dose such as, for example, can be found in KwikPen™ provided by Eli Lilly and Company (Indianapolis, Indiana). FIG. 15 shows such medication delivery device 100′ with a dose button 56, as a single component, and the contact interface 140 (any embodiment disclosed herein) located on the proximal surface of the dose button (shown in dashed lines). Any embodiment of the cover disclosed herein may be coupled with the contact interface 140.

Further details of the design and operation of some embodiments of a delivery device 100 may be found in U.S. Pat. No. 7,195,616, entitled “Medication Injector Apparatus with Drive Assembly that Facilitates Reset,” which is hereby incorporated by reference in its entirety. As discussed above, in some embodiments, the rotatable collar and dose button may be merged into one component. One example of such an arrangement is described in U.S. Pat. No. 7,291,132, entitled “Medication Dispensing Apparatus with Triple Screw Threads for Mechanical Advantage,” which is hereby incorporated by reference in its entirety.

It should be appreciated that in some embodiments, the contact interface 40 may be both axially and rotationally fixed to the dose button 30. In some embodiments, the contact interface 40 may be an extension or portion of the dose button 30, and/or may be attached to the dose button 30. As described previously, the contact interface 40 may be arranged in any suitable way with respect to the dose button 30 to guide a user's finger to axially translate the dose button 30. Accordingly, the contact interface 40 and the dose button 30 may include any connection, interface, or attachment to allow simultaneous movement and/or rotation.

In some embodiments, the contact interface 40 may be attached to a proximal surface 31 of the dose button 30, as shown in FIG. 2, by any suitable means, including, but not limited to, thermal sealing, welding, adhesive bonding, frictional engagement, splines, a snap or press fit, interference fitting, ultrasonic welding, adhesives, a mechanical means, any combinations thereof, or any other suitable means, as the present disclosure is not so limited.

Of course, in some embodiments, the contact interface 40 may be part of the dose button 30, such that the contact interface 40 may be integrally formed with the dose button 30. For example, the contact interface 40 may be co-molded with the dose button 30, or in other examples, the contact interface 40 may be two-shot injection molded with the dose button 30. In some embodiments, the contact interface 40 may be a thin coating of friction enhancing material covering a portion of the dose button 30.

In some embodiments, the contact interface may include a stem, which may be inserted into a lumen of the dose button. The stem may include a rivet-like fixture at an end of the lumen opposite from the contact interface such that the stem (and subsequently the contact interface) may not translate along the lumen and may be axially fixed to the dose button. Of course, embodiments in which the stem of the contact interface and the lumen of the dosing button may be attached together (via interference fitting or ultrasonic welding, or any other suitable attachment mechanism) are also contemplated, as the present disclosure is not so limited. In some embodiments, the lumen of the dose button may serve to help center the contact interface in place.

It should be appreciated that while the contact interface 40 and dose button 30 are shown to be coaxial in FIGS. 1-2, any other non-coaxial arrangement may also be contemplated.

It should be appreciated that combinations of the aforementioned connection schemes between the contact interface 40 and the dose button 30 are also contemplated. For example, the contact interface 40 may be both adhered to a proximal surface 31 of the dose button 30 and may also include a stem inserted into a lumen of the dose button 30. Any suitable connection to axially and rotationally fix the contact interface 40 and the dose button 30 may be used, as the present disclosure is not so limited.

In some embodiments, a medication delivery device may include a data module. The data module may serve one or more functions, such as measuring a delivered dosage, tracking date and time of actuation, and/or measuring other properties of the device. In some embodiments, the data module includes a contact interface. In some embodiments, actuating the contact interface of the data module may also serve to actuate a dose button of the medication delivery device.

One illustrative example of a medication delivery device with a data module is shown in FIGS. 3-6. The medication delivery device 1000 (hereinafter “device”) may include a housing 10 with a proximal portion 11 and a distal portion 13, as described earlier. The device 1000 may also include an outlet 14, from which an injection needle 15 may extend to deliver a medication fluid contained within a cartridge or reservoir located within the distal portion 13. As shown in FIG. 3, the device 1000 may include a data module 250 at an end of the device 1000 opposite from the outlet 14. When the data module 250 is coupled to the device housing 10, the rotating dose member comprises the data module 250 that is rotatable about the longitudinal axis relative to the housing 10 during dose setting and may be rotatable about the longitudinal axis relative to the housing 10 during dose dispensing.

According to some embodiments, the data module 250 (e.g., a dose detection system) may be operable to measure a property of the device 1000 during operation. In some embodiments, the data module 250 may determine information that may correspond to the amount of dose delivered. The determination may be based on relative rotation between a first portion 200 and the housing 10 and/or based on relative rotation between the first portion 200 and a second portion 300. In some embodiments, the data module may include one or more sensor arrangements that serve to detect relative rotation between the first portion 200 and the second portion 300 and/or relative rotation between the data module 250 and the housing 10. In some embodiments, the data module may include one or more sensor arrangement that serve to detect relative rotation between the data module 250 (as a single member-having the first and second portions being integrally formed and not capable for relative rotation therebetween) and the housing 10. According to one aspect, the data module 250 may include a controller to process and communicate output signals from one or more sensors of the module 250 representative of the sensed rotation. In one embodiment, the data module 250 includes an electronics assembly suitable for operation of the sensor arrangement as described herein. The controller is operably connected to the sensor arrangement to receive outputs from one or more rotational sensors. The controller may include conventional components such as a processor, power supply, memory, microcontrollers, etc. contained for example in the body of data module 250. Alternatively, at least some components may be provided separately, such as by means of a computer, smart phone or other device. Means are then provided to operably connect the external controller components with the sensor arrangement at appropriate times, such as by a wired or wireless connection, such as Bluetooth, Wi-Fi, cellular, NFC, or other wireless means.

According to some embodiments of the device 1000, during a dose setting mode of operation, the first portion 200 and the second portion 300 of the data module 250 may be rotationally fixed to the rotatable collar 20 and dose button 30 and may be rotatable relative to the housing 10 and/or the contact interface 40. A user may rotate the first portion 200 and/or the second portion 300 of the data module 250 to set a dose of medication delivery device 1000.

In some embodiments, a user rotates the data module 250 in its entirety relative to the housing 10 and the contact interface 40 to set a dose. In other embodiments, the user rotates only a portion of the data module relative to the housing and the contact interface to set a dose.

In some embodiments, in the dose dispensing mode, the first portion 200 of the data module 250 may be attached to the proximal portion 11 of the housing 10 and may be rotatable relative to the housing 10 about a longitudinal axis AA of the device 1000, as shown in FIG. 3.

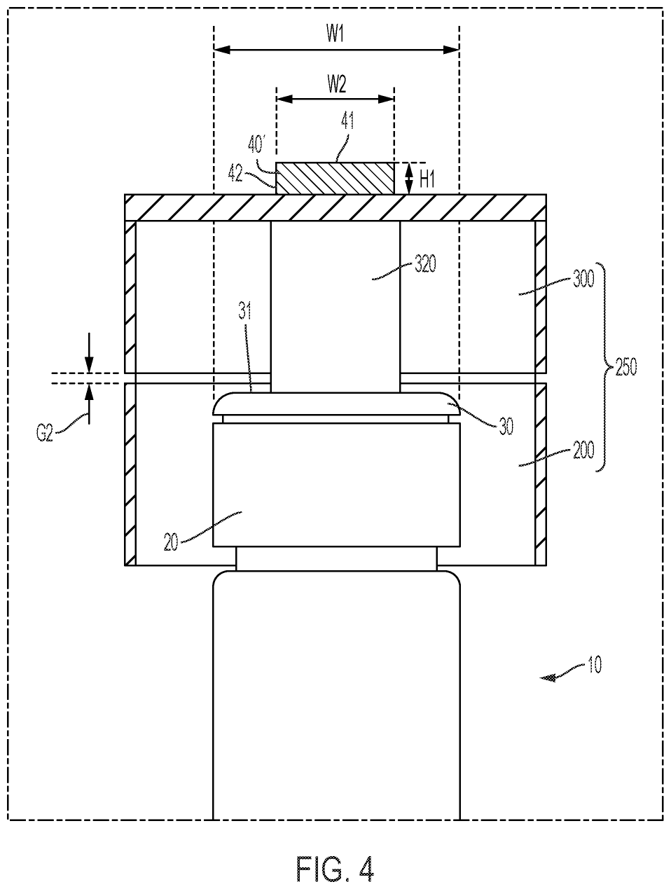

As shown in the cross-section of FIG. 4 taken along line 4-4 of FIG. 3, the data module 250 and contact interface 40 are shown in cross-section. The first portion 200 of the data module 250 may be attached to the rotatable collar 20, such that the two components are rotationally fixed to one another. In other words, a user may manipulate the rotatable collar 20 (to set a dose) or the dose button 30 (to dispense a dose) by manipulating the first portion 200 of the data module 250. In some embodiments, a portion of or the entirety of the rotatable collar 20 and/or the dose button 30 may be located inside the first portion 200. In some embodiments, any portion of the data module 250 may be permanently or removably attached to the device 1000. In some embodiments, the data module may include a contact interface. As shown in FIG. 4, the contact interface 40′ may be mechanically coupled to the dose button 30 such that axial translation of the contact interface 40′ may result in axial translation of the dose button 30.

In some embodiments, the contact interface may be formed of a friction enhancing material. In other embodiments, the contact interface may include a portion formed of a friction enhancing material. For example, the contact interface may be coated with a friction enhancing material or may include one or more features formed of the friction enhancing material.

It should be appreciated that the contact interface 40′ may function as an actuator in some embodiments. In some embodiments, the contact interface 40′ may be attached to a surface or body of the actuator by any suitable means, including, but not limited to, thermal sealing, welding, adhesive bonding, frictional engagement, splines, a snap or press fit, interference fitting, ultrasonic welding, adhesives, a mechanical means, any combinations thereof, or any other suitable means, as the present disclosure is not so limited. In some embodiments, the contact interface 40′ may be part of the actuator, such that the contact interface 40′ may be integrally formed with the actuator. For example, the contact interface 40′ may be co-molded or two-shot injection molded with the actuator. In some embodiments, the contact interface 40′ may be a friction enhancing coating of an actuator which may be attached to or otherwise connected to the data module 250 with any suitable connection schemes. It should be appreciated that combinations of the aforementioned connection schemes between the contact interface 40′ and the actuator are also contemplated. For example, the contact interface 40′ may be a thin coating of friction enhancing material conformally wrapped around a portion (or all of) of the actuator. Any suitable connection to axially and rotationally fix the contact interface 40′ and the actuator may be used, as the present disclosure is not so limited.

As described previously, the contact interface 40′ may guide the user's finger to axially translate the dose button 30. Accordingly, the data module 250 may include one or more intermediate components 320 arranged to connect the dose button 30 to the contact interface 40′ such that axial translation of the contact interface 40′ may cause axial translation (e.g., the user depressing the contact interface 40′) of the dose button 30. The contact interface 40′ and the intermediate component 320 may be axially fixed relative to one another and/or integrally formed into a single component. In other embodiments, any number of intermediate mechanical components 320 may transfer the axial translation of the contact interface 40′ to the dose button 30. In some embodiments, a magnitude of axial translation of the dose button 30 may be substantially equal to a magnitude of axial translation of the contact interface 40′, whereas in other embodiments, the magnitudes of axial translation may differ. In some embodiments, the data module 250 may include one or more intermediate components 320 which may transfer a force from the contact interface 40′ to the dose button 30. In some embodiments, an intermediate component may transfer force to the dose but by abutting against the dose button 30. It should be appreciated that the present disclosure is not limited by the connection scheme between the dose button 30 and the contact interface 40′.

In some embodiments, in the dose dispensing mode, the first portion 200 of the data module 250 may be rotationally fixed to the rotatable collar 20 and rotationally uncoupled from the housing 10. The second portion 300 and contact interface 40′ may be rotationally uncoupled relative to the first portion 200 during dose dispensing operations such that the contact interface 40′ does not rotate with the first portion 200 when a user is operating the device 1000 to dispense a dose. In some embodiments, the contact interface 40′ may be rotationally stationary with respect to the housing 10. As described in further detail above, the contact interface 40′ may include a push surface 41, such that a user may dispense a dose from the device 1000 by axially translating (e.g., pushing) the push surface 41. In some embodiments, the first and second portions are rotationally fixed relative to one another and rotate with the rotatable collar during dose dispensing, relative rotation during dose dispensing is sensed between the first and second portions (as a unit) (or a sensing element associated with the first and/or second portions) and the contact interface 40′/intermediate components 320 (as a unit) (or a sensing element) that are stationary. In some embodiments, the intermediate component houses the sensing element and/or electronic assembly. In this embodiment, during dose dispensing, the sensor may be rotating with the first/second portions and the sensed element is stationary with the contact interface/intermediate components, and alternatively, sensed element may be rotating with the first/second portions and the sensor is stationary with the contact interface/intermediate components. In other embodiments, the second portion 300 may be mechanically coupled to the dose button 30 and axially fixed relative to the first portion 200.

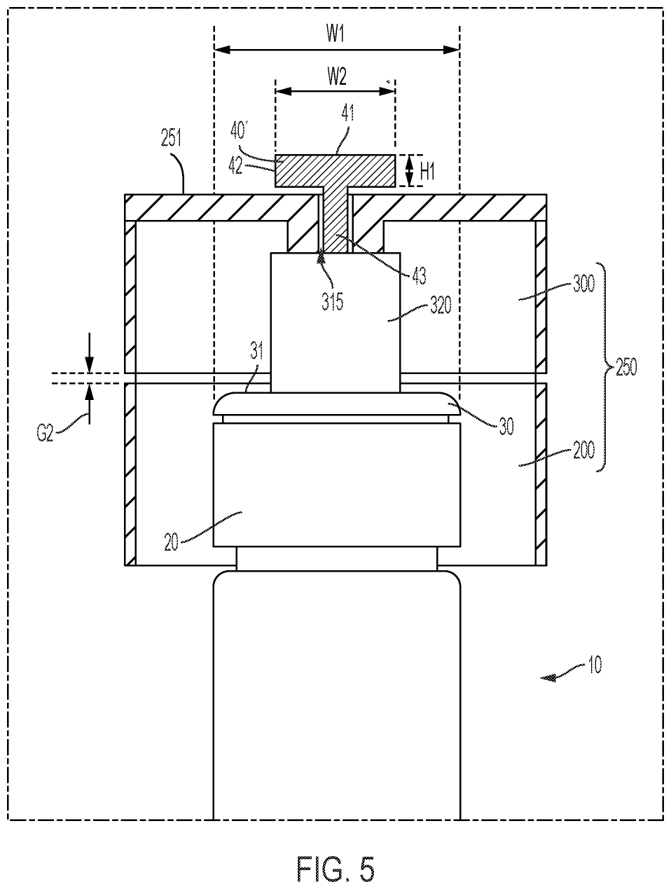

In some embodiments, the contact interface 40′ may be directly attached, adhered, or otherwise affixed to the first portion 200, as shown in FIG. 4. In other embodiments, as shown in FIG. 5, the second portion 300 may include a lumen 315 sized to accept a stem 43 of the contact interface 40′. The stem 43 may be coupled to any one of the intermediate components 320 to translate force applied to the push surface 41 to the dose button 30.

It should be appreciated that while in some embodiments, the stem 43 is formed of the same material as the push surface 41 (e.g., the contact interface 40′ may be formed as one piece), embodiments in which the stem is formed of a different material (e.g., the stem being made of a more rigid material than the push surface 41) are also contemplated. In one example, such as, for example, shown in FIG. 9 and FIG. 10, the stem 943 and a lower portion 941A of the push surface 941 of the contact interface 940 are made from a rigid material, while an upper portion 941B of the contact interface 940 overlying the lower portion 940A of the contact interface is made from a material softer than the rigid material. FIG. 10 is a cross-sectional view of another contact interface 1040 showing the upper portion 1041B of the push surface 1041 formed from softer material sheet that is attached to the lower portion 1041A of the push surface 1041 which is integrally formed with the stem 1043. The lower portion 1041A may include at least one of a radial lip 1045 extending radially beyond the upper portion 1041B and a coupling rim 1047 to which a correspondingly shaped recess 1049 that is formed the confronting surface in the upper portion 1041B is attached such as by thermal bonding or other attachment means.

In some embodiments (not shown), the lumen 315 may extend from the second portion 300 to the dose button 30. In other embodiments, the contact interface 40′ may be attached to a proximal surface 31 of the dose button by any suitable means, including, but not limited to, thermal sealing, welding, adhesive bonding, frictional engagement, splines, a snap or press fit, interference fitting, ultrasonic welding, adhesives, a mechanical means, any combinations thereof, or any other suitable means, as the present disclosure is not so limited. It should be appreciated that the contact interface 40′ may be arranged in any suitable manner with respect to the second portion 300, as the present disclosure is not so limited.

In some embodiments, the contact interface 40, 40′ may include a lateral dimension (e.g., a width) measured along a lateral direction (e.g., along a plane normal to the axial direction of the longitudinal axis AA, as shown in FIGS. 1 and 3). In some embodiments, a dimensional parameter W1 of the proximal surface 31 of the dose button 30 (shown as width) may be greater than a dimensional parameter W2 of the contact interface 40, 40′ (shown as its width). In some embodiments, the width W2 of the contact interface 40, 40′ may be at least 10%, 12%, 15%, 20%, 25%, 30%, 33.33%, 35%, 40%, 45%, 50%, 55%, 60%, 65%, 66.67%, 70%, 75%, 80%, 85%, 90%, 95% of the width W1 of the proximal surface 31 of the dose button, or any other suitable percentage. In other embodiments, the width W2 of the contact interface 40, 40′ may be less than or equal to 95%, 90%, 85%, 80%, 75%, 70%, 66.67%, 65%, 60%, 55%, 45%, 40%, 35%, 33.33%, 30%, 25%, 20%, 15%, 12%, 10% of the width W1 of the proximal surface 31 of the dose button 30, or any other suitable percentage. Combinations of the foregoing ranges are also contemplated. For example, in some embodiments, width W2 of the contact interface 40, 40′ may be 5% to 95%, 10% to 95%, 10% to 90%, 20% to 50%, 33.33% to 66.67%, 50% to 75% of the width W1 of the proximal surface 31, or any other suitable range of percentages. In some embodiments, the width W2 of the contact interface 40, 40′ may be equal to the width W1 of the proximal surface 31. It should be appreciated that the width W2 of the contact interface 40, 40′ measured along a plane normal to a longitudinal axis AA of the device 100 or 1000 may be any suitable percentage of the width W1 of the proximal surface 31, as the present disclosure is not so limited. Although widths W1 and W2 are used herein, the term dimensional parameter that can include surface area, cross-sectional area, contact area, diameter, or the like. Although width W1 is shown relative to the proximal surface 31 of the dose button 30, the width W1 may also be defined relative to the proximal surface 251 of the data module 250.

In other embodiments, the width W2 of the contact interface 40, 40′ may be any size irrespective of the width W1 of the proximal surface 31.

In some embodiments, the width W2 of the contact interface 40, 40′ may be at least 1 mm, 2 mm, 2.5 mm, 3 mm, 3.5 mm, 4 mm, 5 mm, 6 mm, 7.5 mm, 8 mm, 9 mm, 10 mm, 12 mm, 15 mm, 20 mm, or any other suitable width. In some embodiments, the width W2 of the contact interface 40, 40′ may be less than or equal to 20 mm, 15 mm, 12 mm, 10 mm, 9 mm, 8 mm, 7.5 mm, 6 mm, 5 mm, 4 mm, 3.5 mm, 3 mm, 2.5 mm, 2 mm, 1 mm, or any other suitable width. Combinations of the foregoing ranges are also contemplated. For example, in some embodiments, width W2 of the contact interface 40, 40′ may be 1 mm to 20 mm, 2 mm to 10 mm, 2 mm to 5 mm, 2.5 mm to 7.5 mm, 3 mm to 10 mm, 5 mm to 20 mm, or any other suitable range of widths. It should be appreciated that the width W2 of the contact interface 40, 40′ measured along a plane normal to a longitudinal axis AA of the device 100 or 1000 may be any suitable width, as the present disclosure is not so limited.

As shown in FIGS. 2, 4, and 5, in some embodiments, the contact interface 40, 40′ may extend from either the dose button 30 or the second portion 300 by a height H1. In other words, the contact interface 40, 40′ may protrude from the dose button 30 or the second portion 300. The height H1, as shown in FIGS. 2, 4, and 5, may be any suitable height to allow ergonomic operation of the device 100 or 1000. In some embodiments, the height H1 may be at least 0.02 mm, 0.05 mm, 0.1 mm, 0.2 mm, 0.3 mm, 0.4 mm, 0.5 mm, 0.6 mm, 0.8 mm, 1 mm, 1.2 mm, 1.4 mm, 1.5 mm, 1.6 mm, 1.8 mm, 2 mm, 2.5 mm, 3 mm, 3.5 mm, 4 mm, 5 mm, 7 mm, or any other suitable height. In other embodiments, the height H1 may be less than or equal to 7 mm, 5 mm, 4 mm, 3.5 mm, 3 mm, 2.5 mm, 2 mm, 1.8 mm, 1.6 mm, 1.5 mm, 1.4 mm, 1.2 mm, 1 mm, 0.8 mm, 0.6 mm, 0.5 mm, 0.4 mm, 0.3 mm, 0.2 mm, 0.1 mm, 0.05 mm, 0.02 mm, or any other suitable height. Combinations of the foregoing ranges are also contemplated. For example, in some embodiments, the height H1 may be 0.02 mm to 7 mm, 0.05 mm to 5 mm, 0.1 mm to 2 mm, 0.1 mm to 1 mm, or any other suitable range. It should be appreciated that the height H1 of the contact interface 40, 40′ measured along a longitudinal axis AA of the device 100 or 1000 may be any suitable height, as the present disclosure is not so limited.

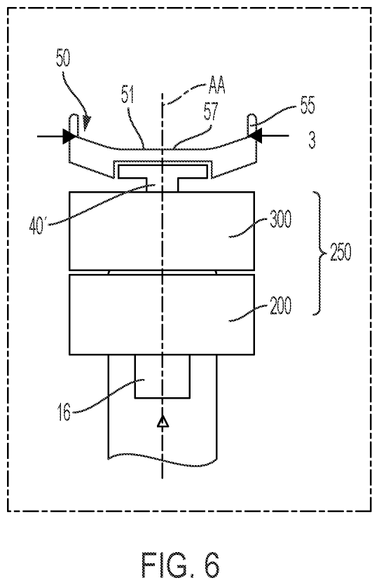

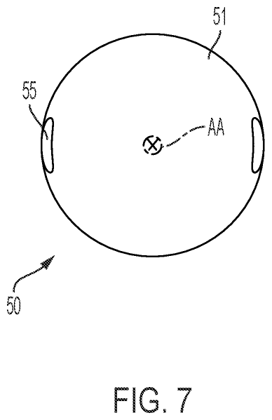

In some embodiments, a medication delivery device may include an actuator cover. As shown in FIG. 6, actuator cover 50 may be coupled to the contact interface 40′ such that the user may interact with the cover 50 to operate the device (e.g., to dispense medication). The cover 50 may include a push surface 51 and sidewalls 55. In embodiments where the medication delivery device includes a cover 50, the user may actuate the device by pushing the push surface 51 of the cover 50. The sidewalls 55 may extend from the push surface 51 in a direction away from the housing 10, as shown in FIGS. 6 and 7. In some embodiments, the sidewalls 55 may help to guide a user's finger along the push surface 51. The sidewalls 55 may help to reduce the likelihood of off-center sliding of the user's finger along the push surface 51. The cover 50 may extend along a longitudinal axis AA such that the push surface 51 may be further away from the data module 250 than the contact interface 40′. In some embodiments, the user's finger may be less likely to interact with the rotating dose component, such as, for example, the data module 250, given the greater axial distance or gap between the push surface 51 and the data module 250. In some embodiments, a user's finger sliding or resting between the sidewalls 55 may be less likely to rotate the cover 50, and subsequently the contact interface 40′ and other components of the medication delivery device. In this way, the sidewalls 55 may help to reduce accidental rotation of the rotatable collar 20 or any other dose-setting component. The cover 50 may contribute to isolating the applied force through the cover and contact interface and away from the proximal surface of the rotating dose component. In one embodiment, the sidewall 1155 in the cover 1150 shown in FIG. 11 may form a singular, arcuate sidewall to define a physical stop for the patient's finger. In another embodiment, the sidewall 1255 in the cover 1250 shown in FIG. 12 may define a U-shaped sidewall to define a physical stop for the patient's finger and to provide additional contact face for the patient in comparison to the cover 1250 with portions 1250A and 1250B extending radially beyond the cross-sectional area of the actuator (as defined by the dashed lines).

In some embodiments, the actuator cover 50 may include a cover recess 57 to accommodate a user's finger, as shown in FIG. 6. The cover recess may be positioned centrally on the actuator cover 50 such that the push surface 51 may be symmetrically recessed around the axis AA. In some embodiments, the actuator cover 50 may be formed as a saddle shape (as shown in FIG. 6), such that the push surface 51 may be curved. In some embodiments, a lowest portion of the push surface may be positioned along the longitudinal axis AA. In some embodiments, the user's finger may be guided to the center of the cover 50 both due to the curvature of the push surface 51 and the sidewalls 55. In some embodiments, the sidewalls 55 may extend parallel to the longitudinal axis AA, as shown in FIG. 7. In other embodiments, the sidewalls may be angled with respect to the axis AA. In some embodiments, the sidewalls may be curved for comfortable and/or ergonomic operation by the user. For example, the sidewalls 55 may be oval-shaped, as shown in FIG. 7. In some embodiments, the sidewalls 55 may have ridges. The ridges may help to confine the user's finger on the push surface 51. It should be appreciated that the sidewalls may be any suitable shape to guide a user's finger along the push surface, as the present disclosure is not so limited.

Of course, embodiments in which the push surface is generally flat are also contemplated. The actuator cover may have sidewalls that may help constrict movement of the user's finger on the cover to help avoid off-center sliding. The push surface 51 may have any suitable shape (e.g., hemispherical, polygonal, flat, curved, etc.) to guide a user's finger. In some embodiments, the push surface 51 may have rounded or chamfered edges for comfortable and/or ergonomic operation by the user. The present disclosure is not limited by the surface shape of the push surface 51. As shown in FIG. 6, the width of the contact interface 40′ may still be width W2, however the dimensional aspect or width W3 of cover 50 may be greater than the width W1 of the contact surface and may be about the same as the width W1 of the proximal surface of the rotating dose component, such as, for example, the data module.

The actuator cover 50, as shown in FIGS. 6 and 7, may be formed of any suitable material, including a rigid material or a compliant material. In some embodiments, the cover may be formed of a combination of materials, e.g., a first material and a second material that is more compliant than the first material. In some embodiments, the entire cover 50 may be formed of a compliant material. In other embodiments, the entire cover 50 may be formed of a rigid material. It should be appreciated that the current disclosure is not limited by the material composition of the actuator cover 50. In some embodiments, the cover may be formed of a rigid material, and a non-rigid material, such as an elastomer, layer may be applied to the push surface 51.

In some embodiments, the actuator cover 50 may snap-fit onto the contact interface 40′. In some embodiments, the actuator cover 50 is permanently attached to the contact interface 40′. In other embodiments, the actuator cover 50 may removably attach to the contact interface 40′. The cover 50 may be detached from the contact interface 40′ via any suitable action, e.g., twisting, pulling, sliding, squeezing, etc. the cover 50 off of the contact interface 40′. It should be appreciated that the direction and magnitude of force required to attach/detach the cover may be distinct from the direction and/or magnitude of force required to actuate the medication delivery device. This may help to avoid setting and/or dispensing a dose when the cover is being attached/detached. The cover may be coupled to the contact interface with any suitable attachment mechanism (e.g., snap-fit, threaded attachment, magnetic, twist-lock, adhesives, etc.) to allow a user to attach/detach the cover from the contact interface. In some embodiments, the cover may be formed integrally with the contact interface into a single-piece component, such as being molded, such that detachment of the cover from the contact interface may not be possible.

The contact interface 40, 40′ may be any suitable shape to allow a user to axially displace (e.g., translate) the dose button 30 to operate the device 100 or 1000 in the dose dispensing mode. For example, the contact interface 40, 40′ may be cylindrical such that it may include a sidewall 42 spanning a periphery of the interface 40, 40′, as shown in FIGS. 2, 4, and 5. In some embodiments, the sidewall 42 may be perpendicular to the proximal surface 31 of the dose button, as shown in FIG. 2, whereas in other embodiments, the sidewall 42 may be angled with respect to the proximal surface 31. For example, the contact interface 40, 40′ may be tapered. As an illustrative example, the contact interface may be tapered such that the sidewall 42 may be angled at 45 degrees, or at any other suitable angle, with respect to the proximal surface 31. FIG. 13 shows an example of the contact interface 1340 having a tapered sidewall 1342 leading to the proximal surface such that the cross-sectional area is increasingly smaller moving in the proximal direction from the distal end. It should be appreciated that the sidewall 42 may be tapered at any suitable angle towards or away from the longitudinal axis AA, as the present disclosure is not so limited. In some embodiments, the contact interface 40, 40′ may be curved such that the sidewall 42 may include a non-linear slope with respect to the proximal surface 31. For example, a portion of or the entirety of the contact interface 40, 40′ may be dome-shaped. It should be appreciated that any suitable shape of the contact interface 40, 40′ and/or push surface 41 may be used, as the present disclosure is not so limited.

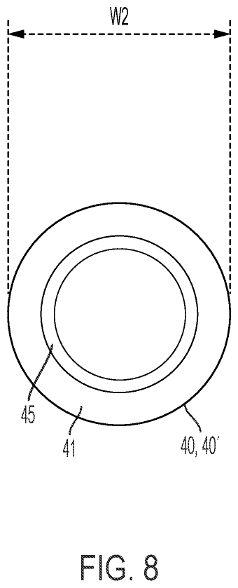

In some embodiments, the contact interface 40, 40′ may include one or more features to serve as a guide to center a user's finger on the interface 40, 40′ and may allow for ergonomic operation of the interface 40, 40′. In some embodiments, the contact interface 40, 40′ may include one or more protrusions. Such protrusions may help to enhance grip between the contact interface 40, 40′ and the user's finger. The protrusions may be shaped as ribs, circles, squares, zigzags, waves, or any other suitable shape. In the illustrative embodiment shown in FIG. 8, which depicts a top view of a contact interface, the contact interface 40, 40′ includes one or more circular rib 45 that protrude out of the push surface 41 of the contact interface. In some embodiments, the ribs may extend radially outwardly, such as, for example, the ribs 1345 of the contact interface 1340 in FIG. 13. It should be appreciated that non-radial arrangements of ribs or any other suitable protrusions on the contact interface 40, 40′ are also contemplated, as the present disclosure is not limited by the surface structure of the contact interface 40, 40′. For example, protrusions may be spread out over an area on the push surface 41, may be arranged in one or more circles or other loops, or any other suitable arrangement.

In some embodiments, the contact interface 40, 40′ may include a contact interface recess (such as, for example, recess 46 or 946) along the push surface 41 for ergonomic operation. The contact interface recess may be any suitable shape (e.g., hemispherical, curved, cylindrical, conical, etc.), as the present disclosure is not limited by the structure of the contact interface 40, 40′. In some embodiments, the contact interface recess may extend radially from the sidewall 42 of the contact interface 40, 40′ to the longitudinal axis AA. In other embodiments, the contact interface recess may extend partially radially from the sidewall 42 of the contact interface 40, 40′ to the longitudinal axis AA. In other embodiments still, the push surface 41 may include more than one contact interface recess to enhance the friction between the contact interface 40, 40′ and the user. For example, the push surface 41 may include a one or more circular contact interface recesses or protrusions to stabilize a user's finger and reduce the likelihood of undesirable rotation of the dose button 30. In some embodiments, the features and/or structure of the push surface 41 may be for both ergonomic and aesthetic purposes. For example, the sidewall 42 of the contact interface 40, 40′ may include chamfered or rounded edges.

It should be appreciated that a contact interface 40, 40′ according to some embodiments may include a combination of the features listed above, as the present disclosure is not so limited. For example, the ribs of the push surface 41 may include a plurality of radially distributed ribs protruding from a concave push surface 41 (e.g., the push surface 41 may be recessed into the contact interface 40, 40′). In another example, the sidewall 42 of the contact interface 40, 40′ may be tapered with respect to the proximal surface 31 and may include a plurality of ribs radially distributed along the sidewall 42. In another example, the ribs may be aligned in parallel to one another across the push surface 41 or surface 51. FIG. 14 shows an example of the cover 1450 having a plurality of ribs 1445 extending across the surface 1451 in a parallel arrangement.

The dose detection system uses a sensing component and a sensed component. One of these components may be coupled (directly or indirectly) to members of the medication delivery device. Various sensor systems are contemplated herein. The term “sensing component” refers to any component which is able to detect the relative position of the sensed component. The sensing component includes a sensing element, or “sensor”, along with associated electrical components to operate the sensing element. The “sensed component” is any component for which the sensing component is able to detect the position and/or movement of the sensed component relative to the sensing component. For the dose delivery detection system, one of the sensed component or the sensing component rotates relative to the other, which is able to detect the angular position and/or the rotational movement of the rotating sensed component or sensing component. The sensing component may comprise one or more sensing elements, and the sensed component may comprise one or more sensed elements. The sensor system is able to detect the position or movement of the sensed component(s) and to provide outputs representative of the position(s) or movement(s) of the sensed component(s). Sensing and determining data may occur prior to dose setting, during dose setting, during dose delivery, or after dose delivery. Information may include time/date, dose set amount, dose delivered amount, product identification data, battery life remaining, errors codes, as well as other information about the operation of the device.

A sensor system typically detects a characteristic of a sensed parameter which varies in relationship to the position of the one or more sensed elements within a sensed area. The sensed elements extend into or otherwise influence the sensed area in a manner that directly or indirectly affects the characteristic of the sensed parameter. The relative positions of the sensor and the sensed element affect the characteristics of the sensed parameter, allowing the controller of the sensor system to determine different positions of the sensed element. Suitable sensor systems may include the combination of an active component and a passive component. With the sensing component operating as the active component, it is not necessary to have both components connected with other system elements such as a power supply or controller.

Any one of a variety of sensing technologies may be incorporated by which the relative positions of two members can be detected. Such technologies may include, for example, technologies based on tactile, optical, magnetic, acoustical, inductive or electrical measurements.

In one aspect, the sensor system detects relative positions or movements of the rotating sensed elements or sensing elements, and therefore of the associated members of the medication delivery device. The sensor system produces outputs representative of the position(s) or the amount such movement. For example, the sensor system may be operable to generate outputs by which the rotation of the rotating dose member during dose delivery can be determined. A controller is operably connected to each sensor to receive the outputs. In one aspect, the controller may be configured to determine from the outputs the amount of dose delivered by operation of the medication delivery device. In another aspect, the controller may be configured to determine from the outputs data that may be used to determine the amount of dose delivered by operation of the medication delivery device.

With the extent of rotation having a known relationship to the amount of a delivered dose, the sensor system operates to detect the amount of angular movement from the start of a dose injection to the end of the dose injection. For example, a typical relationship for a pen injector is that an angular displacement of a rotating dose member of 18° is the equivalent of one unit of dose, although other angular relationships are also suitable. The sensor system is operable to determine the total angular displacement of a rotating dose member during dose delivery. Thus, if the angular displacement is 90°, then 5 units of dose have been delivered. One approach for detecting the angular displacement is to count increments of dose amounts as the injection proceeds. For example, a sensor system may use a repeating pattern of sensed elements, such that each repetition is an indication of a predetermined degree of angular rotation. Conveniently, the pattern may be established such that each repetition corresponds to the minimum increment of dose that can be set with the medication delivery device.

An alternative approach is to detect the start and stop positions of the relatively moving member, and to determine the amount of delivered dose as the difference between those positions. In this approach, it may be a part of the determination that the sensor system detects the number of full rotations of the rotating dose member. Various methods for this are well within the ordinary skill in the art and may include “counting” the number of increments to assess the number of full rotations.

The sensor system components may be permanently or removably attached to the medication delivery device. In an illustrative embodiment, as least some of the dose detection system components are provided in the form of a module that is removably attached to the medication delivery device. This has the advantage of making these sensor components available for use on more than one pen injector.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Various aspects are described in this disclosure, which include, but are not limited to, the following aspects:

1. A medication delivery device including: a housing disposed about a longitudinal axis and having an outlet; a rotating dose member rotatable about the longitudinal axis relative to the housing during dose setting; a dose button configured to be translatable along the longitudinal axis in an axial direction relative to the housing to activate a dose dispensing mode in which medication is dispensed out of the outlet, the dose button including a proximal surface; and a contact interface disposed proximal to and configured to contact the proximal surface of the dose button, the contact surface having a proximal surface, the contact interface and the dose button having a coaxial relationship, wherein the proximal surface of the dose button includes a first dimensional parameter, the proximal surface of the contact interface includes a second dimensional parameter that is smaller than the first dimensional parameter, the second dimensional parameter sized to enhance on-center axial loading of the contact surface during dose delivery and inhibit any axial loading on the rotating dose member.

2. The medication delivery device of aspect 1, wherein the rotating dose member further includes a collar rotatably mounted relative to the housing, wherein rotation of the collar relative to the housing sets an amount of medication to be dispensed out of the outlet during the dose dispensing mode, and wherein the collar rotates relative to the dose button during actuation of the dose button.

3. The medication delivery device of aspect 1, wherein the dose button is rotatably mounted relative to the housing, wherein rotation of the dose button relative to the housing sets an amount of medication to be dispensed out of the outlet during the dose dispensing mode.

4. A. medication delivery device including: a housing having an outlet; a dose button configured to be translatable in an axial direction relative to the housing to activate a dose dispensing mode in which medication is dispensed out of the outlet, the dose button including a proximal surface; and a data module configured to measure a property in the dose dispensing mode, the data module having a contact interface, and the data module being operatively coupled to the dose button, the data module including a proximal surface, wherein a first lateral dimension measured across the proximal surface of the data module in a lateral direction is greater than a second lateral dimension measured across a proximal surface of the contact interface in the lateral direction, the lateral direction being perpendicular to the axial direction.

5. The medication delivery device of aspect 4, further including a collar rotatably mounted relative to the housing, wherein rotation of the collar relative to the housing sets an amount of medication to be dispensed out of the outlet during the dose dispensing mode, and wherein the collar rotates relative to the dose button during actuation of the dose button.

6. The medication delivery device of aspect 5, wherein the data module is configured to rotate relative to the contact interface during the dose dispensing mode.

7. The medication delivery device of aspect 6, wherein the data module includes a first portion and a second portion rotationally fixed relative to one another, wherein the first portion and the second portion are rotatably fixed to the collar, and wherein the contact interface is rotationally fixed to the housing, and the first portion and the second portion rotate relative to the contact interface during the dose dispensing mode. The contact interface is rotationally fixed to the second portion of the data module.

8. The medication delivery device of aspect 6, wherein the data module includes a first portion and a second portion, wherein the first portion is rotatably fixed to the collar, and wherein the contact interface is rotationally fixed to the housing, the first portion and the second portion rotate relative to one another during the dose dispensing mode.

9. The medication delivery device of aspect 4, wherein the dose button is rotatably mounted relative to the housing, wherein rotation of the dose button relative to the housing sets an amount of the medication to be dispensed out of the outlet during the dose dispensing mode.

10. The medication delivery device of aspect 9, wherein the data module is configured to rotate relative to the contact interface during the dose dispensing mode.

11. The medication delivery device of any one of the above aspects, wherein the second lateral dimension is between one-third to two-thirds of the first lateral dimension.

12. The medication delivery device of any one of the above aspects, wherein the contact interface includes one or more protrusions extending radially outwardly.

13. The medication delivery device of any one of the above aspects, further including an intermediate component housing a sensing element, wherein the contact interface and the intermediate component are axially fixed relative to one another.

14. The medication delivery device of any one of the above aspects, wherein the contact interface includes a contact interface recess positioned centrally to the contact interface.

15. The medication delivery device of any one of aspects 4-14, further including an actuator cover coupled to the contact interface.

16. The medication delivery device of aspect 15, wherein the actuator cover includes a cover recess positioned centrally to the actuator cover.

17. The medication delivery device of aspect 16, wherein the actuator cover includes at least two sidewalls, and wherein each of the at least two sidewalls are positioned on opposing sides of the cover recess.

18. The medication delivery device of aspect 16, wherein the cover recess is saddle-shaped.

19. The medication delivery device of any one of the above aspects, wherein the dose button is made of a first material and the contact interface is made of a second material, the first material having a greater Young's modulus than a Young's modulus of the second material.

20. The medication delivery device of aspect 17, wherein a coefficient of kinetic friction between the second material and a user's finger is greater than a coefficient of kinetic friction between the first material and the user's finger.

21. The medication delivery device of any one of aspects 19-20, wherein the second material is an elastomeric material.

22. The medication delivery device of any one of aspects 1-21, wherein the housing includes a reservoir configured to hold a medication.

23. A method of delivering medication including: applying an axial force to a contact interface operably coupled to a proximal surface of a dose button; displacing the dose button relative to a housing in an axial direction; and activating a dose dispensing mode in which a medication is dispensed out of an outlet with the displacement of the dose button; wherein a first lateral dimension measured across the proximal surface of the dose button in a lateral direction is greater than a second lateral dimension measured across a proximal surface of the contact interface in the lateral direction, the lateral direction being perpendicular to the axial direction.

24. The method of aspect 23, wherein the contact interface is attached to the proximal surface of the dose button.

25. The method of any one of aspects 23-24, further including measuring a property during the dose dispensing mode with a data module, the data module including the contact interface.

26. The method of any one of aspects 23-25, further including rotating a collar relative to the housing to set an amount of medication to be dispensed out of the outlet in a dose setting mode, and wherein the collar rotates relative to the dose button during the dose dispensing mode.

27. The method of aspect 26, further including measuring a property during the dose dispensing mode with a data module, the data module including the contact interface, wherein the data module includes a first portion and a second portion, wherein in the dose dispensing mode, the first portion is rotatably fixed to the collar and the second portion is rotatably fixed to the housing, and wherein the contact interface is rotationally fixed to the second portion.

28. The method of any one of aspects 23-27, further including rotating the dose button relative to the housing to set an amount of medication to be dispensed out of the outlet during the dose dispensing mode.

29. The method of any one of aspects 23-28, further including installing an actuator cover on the contact interface, wherein the actuator cover includes at least two sidewalls and a cover recess positioned centrally to the actuator cover, and wherein each of the at least two sidewalls are positioned on opposing sides of the recess.

30. The method of any one of aspects 23-29, wherein the dose button is made of a first material and the contact interface is made of a second material, the first material having a greater Young's modulus than a Young's modulus of the second material.

1-3. (canceled)

4. A medication delivery device comprising:
a housing having an outlet at a distal end of the housing;
a dose button at a proximal end of the housing and configured to be translatable in an axial direction relative to the housing to activate a dose dispensing mode in which a medication is dispensed out of the outlet, the dose button including a proximal surface;
a data module configured to measure a property in the dose dispensing mode, the data module having a contact interface protruding from a proximal surface of the data module, and the data module being operatively coupled to the dose button, the data module comprising a proximal surface; and
an actuator cover coupled to the contact interface,
wherein a first lateral dimension measured across the proximal surface of the data module in a lateral direction is greater than a second lateral dimension measured across a proximal surface of the contact interface in the lateral direction, the lateral direction being perpendicular to the axial direction, and
wherein the actuator cover is configured to isolate an applied force for activation of the dose dispensing mode through the actuator cover and the contact interface and away from the proximal surface of the data module.

5. The medication delivery device of claim 4, further comprising a collar rotatably mounted relative to the housing, wherein rotation of the collar relative to the housing sets an amount of the medication to be dispensed out of the outlet during the dose dispensing mode, and wherein the collar rotates relative to the dose button during actuation of the dose button.

6. The medication delivery device of claim 5, wherein the data module is configured to rotate relative to the contact interface during the dose dispensing mode.

7. The medication delivery device of claim 6, wherein the data module includes a first portion and a second portion rotationally fixed relative to one another, wherein the first portion and the second portion are rotatably fixed to the collar, and wherein the contact interface is rotationally fixed to the housing and the first portion and the second portion rotate relative to the contact interface during the dose dispensing mode.

8. The medication delivery device of claim 6, wherein the data module includes a first portion and a second portion, wherein the first portion is rotatably fixed to the collar, and wherein the contact interface is rotationally fixed to the housing, the first portion and the second portion rotate relative to one another during the dose dispensing mode.

9. The medication delivery device of claim 4, wherein the dose button is rotatably mounted relative to the housing, wherein rotation of the dose button relative to the housing sets an amount of the medication to be dispensed out of the outlet during the dose dispensing mode.

10. The medication delivery device of claim 9, wherein the data module is configured to rotate relative to the contact interface during the dose dispensing mode.

11. The medication delivery device of claim 4, wherein the second lateral dimension is between one-third to two-thirds of the first lateral dimension.

12. The medication delivery device of claim 4, wherein the contact interface includes one or more protrusions extending radially outwardly.

13. The medication delivery device of claim 4, further comprising an intermediate component housing a sensing element, wherein the contact interface and the intermediate component are axially fixed relative to one another.

14. The medication delivery device of claim 4, wherein the contact interface includes a contact interface recess positioned centrally to the contact interface.

15. The medication delivery device of claim 4, further comprising an actuator cover coupled to the contact interface.

16. The medication delivery device of claim 15, wherein the actuator cover includes a cover recess positioned centrally to the actuator cover.

17. The medication delivery device of claim 16, wherein the actuator cover comprises at least two sidewalls, and wherein each of the at least two sidewalls are positioned on opposing sides of the cover recess.

18. The medication delivery device of claim 16, wherein the cover recess is saddle-shaped.

19. The medication delivery device of claim 4, wherein the dose button is made of a first material and the contact interface is made of a second material, the first material having a greater Young's modulus than a Young's modulus of the second material.

20. The medication delivery device of claim 19, wherein a coefficient of kinetic friction between the second material and a user's finger is greater than a coefficient of kinetic friction between the first material and the user's finger.

21. The medication delivery device of claim 19, wherein the second material is an elastomeric material.

22. The medication delivery device of claim 4, wherein the housing includes a reservoir including a medication.

23-30. (canceled)

31. A medication delivery device comprising:
a housing having an outlet at a distal end of the housing, a dose button at a proximal end of the housing, a collar rotatably mounted relative to the housing, wherein rotation of the collar relative to the housing sets an amount of a medication to be dispensed out of the outlet during a dose dispensing mode, and wherein the collar rotates relative to the dose button during actuation of the dose button, the dose button configured to be translatable in an axial direction relative to the housing to activate the dose dispensing mode in which the medication is dispensed out of the outlet, the dose button including a proximal surface;
a data module configured to measure a property in the dose dispensing mode, the data module having a contact interface protruding from a proximal surface of the data module, the contact interface configured to rotate relative to at least a portion of the data module during the dose dispensing mode, and the data module being operatively coupled to the dose button and to the collar, the data module comprising a proximal surface; and
an actuator cover coupled to the contact interface,
wherein a first lateral dimension measured across the proximal surface of the data module in a lateral direction is greater than a second lateral dimension measured across a proximal surface of the contact interface in the lateral direction, the lateral direction being perpendicular to the axial direction, and
wherein the actuator cover is sized and shaped to isolate an applied force for activation of the dose dispensing mode away from the proximal surface of the data module.
